(19) United States
(12) Patent Application Publication
(10) Pub. No.: US 2020/0210689 A1
(43) Pub. Date: Jul. 2, 2020

(54) A SYSTEM AND A METHOD FOR ANALYZING A BEHAVIOR OR AN ACTIVITY OF AN OBJECT

(71) Applicant: Mindfio Limited, Central (HK)

(72) Inventor: Jarm Yan Jamnan Law, Quarry Bay (HK)

(21) Appl. No.: 16/643,081

(22) PCT Filed: Oct. 9, 2018
(86) PCT No.: PCT/CN2018/109505

§ 371 Date: Feb. 28, 2020

(30) Foreign Application Priority Data
Oct. 20, 2017 (HK) 17110688.7

(51) Int. Cl. G06K 9/00; G06T 7/20; A61B 5/0205

(52) U.S. Cl. CPC G06K 9/00335; A61B 5/0205; G06T 7/20; G06K 9/00201

(57) ABSTRACT

A system and a method for analyzing a behavior or an activity of an object comprising the steps of obtaining movement data associated with a motion of the object for a predetermined period of time; processing the movement data to obtain physiological parameters associated with the motion of the object; and determining a behavior or an activity of the object based on the obtained physiological parameters over the predetermined period of time.

### TECHNICAL FIELD

The present invention relates to a system and a method for analyzing a behavior or an activity of an object, and particularly, although not exclusively, to a smart wristband for measuring physiological parameters.

### BACKGROUND

Physiological parameter may be measured by using a special electronic device worn near a particular organ. For example, breathing rate is typically measured by using a chest-belt or belly-belt or a device worn near the chest or waist. These measuring devices are not usually worn continuously due to their inconvenient and uncomfortable form-factors. On the other hand, the smart wristband or the smart terminal is a well-acceptable form-factor and is proven to be convenient, which means users are comfortable to wear smart wristbands or carry smart terminal continuously during daily activities. The invention aims to measure physiological parameters from the wristband or the smart terminal so that the data can be continuous measured conveniently and comfortably.

### SUMMARY OF THE INVENTION

In accordance with a first aspect of the present invention, there is provided a method for analyzing a behavior or an activity of an object, comprising the steps of: obtaining movement data associated with a motion of the object for a predetermined period of time; processing the movement data to obtain physiological parameters associated with the motion of the object; and determining a behavior or an activity of the object based on the obtained physiological parameters over the predetermined period of time.

In an embodiment of the first aspect, the physiological parameters include an instantaneous breathing rate of the object.

In an embodiment of the first aspect, the motion of the object includes a body movement as a result of breathing of the object.

In an embodiment of the first aspect, the motion of the object is represented as an inhale-to-exhale waveform associated with the movement data.

In an embodiment of the first aspect, the inhale-to-exhale waveform includes peaks and valleys each representing an inhalation and an exhalation respectively.

In an embodiment of the first aspect, the method further comprises the step of detecting the motion of the object using at least one motion sensor.

In an embodiment of the first aspect, the at least one motion sensor includes an accelerometer and/or a gyroscope.

In an embodiment of the first aspect, the at least one motion sensor includes a 3-axis accelerometer and/or a 3-axis gyroscope.

In an embodiment of the first aspect, the method further comprises the step of processing at least one source signal obtained by the at least one motion sensor associated with the detected motion to obtain the physiological parameters.

In an embodiment of the first aspect, the step of processing the at least one source signal includes filtering a noise signal from the at least one source signal acquired by the motion sensor.

In an embodiment of the first aspect, the noise signal is associated with a quality index of the at least one source signal acquired by the motion sensor.

In an embodiment of the first aspect, the method further comprises the step of consolidating source signals associated with the detected motion obtained in a plurality of axes to obtain the movement data.

In an embodiment of the first aspect, the physiological parameters further include a heart rate and/or a heart rate variability of the object.

In accordance with a second aspect of the present invention, there is provided a system for analyzing a behavior or an activity of an object, comprising: a motion detection module arranged to obtain movement data associated with a motion of the object for a predetermined period of time; and a processing module arranged to process the movement data to obtain physiological parameters associated with the motion of the object, wherein the processing module is further arranged to determine a behavior or an activity of the object based on the obtained physiological parameters over the predetermined period of time.

In an embodiment of the second aspect, the physiological parameters include an instantaneous breathing rate of the object.

In an embodiment of the second aspect, the motion of the object includes a body movement as a result of breathing of the object.

In an embodiment of the second aspect, the motion of the object is represented as an inhale-to-exhale waveform associated with the movement data.

In an embodiment of the second aspect, the inhale-to-exhale waveform includes peaks and valleys each representing an inhalation and an exhalation respectively.

In an embodiment of the second aspect, the motion detection module comprises at least one motion sensor.

In an embodiment of the second aspect, the at least one motion sensor includes an accelerometer and/or a gyroscope.

In an embodiment of the second aspect, the at least one motion sensor includes a multi-axis accelerometer and/or a multi-axis gyroscope.

In an embodiment of the second aspect, at least one motion sensor includes a high-precision multi-axis accelerometer.

In an embodiment of the second aspect, the high-precision multi-axis accelerometer is arranged to sample the motion of the object with a sampling rate of at least 25 Hz.

In an embodiment of the second aspect, the high-precision multi-axis accelerometer is arranged to sample the motion of the object with a resolution of at least 12-bit.

In an embodiment of the second aspect, the processing module is further arranged to process at least one source signal obtained by the at least one motion sensor associated with the detected motion to obtain the physiological parameters.

In an embodiment of the second aspect, the processing module is further arranged to filter a noise signal from the at least one source signal acquired by the motion sensor.

In an embodiment of the second aspect, the noise signal is associated with a quality index of the at least one source signal acquired by the motion sensor.

In an embodiment of the second aspect, the processing module is further arranged to consolidating source signals associated with the detected motion obtained in a plurality of axes to obtain the movement data.

In an embodiment of the second aspect, the physiological parameters further include a heart rate and/or a heart rate variability of the object.

In an embodiment of the second aspect, the system further comprises an engagement means arranged to engage the motion detection module to an object or a user.

In an embodiment of the second aspect, the motion detection module is provided in a wearable device.

In an embodiment of the second aspect, the system further comprises a communication module arranged to communicate the movement data to an external device including the processing module.

In an embodiment of the second aspect, the system further comprises a storage module arranged to at least temporally store the movement data obtained by the motion detection module.

In an embodiment of the second aspect, the communication module includes a Bluetooth communication module.

In an embodiment of the second aspect, the processing module includes a digital signal processor.

In accordance with a third aspect of the present invention, there is provided a smart wristband for measuring physiological parameters, comprising a set of motion sensors for measuring movement of a user's body, at least one processor, at least one storage system in signal communication with the processor, one or more programs stored in the storage system and executable by the processor, the one or more programs comprising instructions for: obtaining movement data by using the set of motion sensors; transforming the movement data to physiological signal; obtaining instantaneous physiological parameters according to physiological parameters according to physiological signal further comprises: determining instantaneous inhale-to-exhale, heart rate, heart rate variability according to physiological signal.

In an embodiment of the third aspect, obtaining instantaneous physiological parameters according to physiological signal further comprises: determining instantaneous inhale-to-exhale according to physiological signal.

In an embodiment of the third aspect, the set of motion sensors includes one or more 3-axis accelerometers.

In an embodiment of the third aspect, obtaining movement data by using the set of motion sensors further comprises: obtaining movement data by reading output data from the 3-axis accelerometers.

In an embodiment of the third aspect, transforming the movement data to physiological signal further comprises: transforming 3-axis accelerometers' output data to physiological signal.

In an embodiment of the third aspect, obtaining instantaneous physiological parameters according to physiological signal further comprises: transforming the physiological signal to instantaneous physiological parameters by using digital signal processing techniques.

In an embodiment of the third aspect, transforming 3-axis accelerometers' output data to physiological signal further comprises: transforming 3-axis accelerometers' output data to inhale-to-exhale waveform, preferably by using a predetermined transformation process, which includes processing a quality index that can be used to distinguish noisy output data with good output data.

In an embodiment of the third aspect, after obtaining instantaneous physiological parameters according to physiological signal, the wristband further comprises instructions for: sending the instantaneous physiological parameters to health-related applications.

In an embodiment of the third aspect, obtaining movement data by reading output data from the 3-axis accelerometers further comprises: obtaining accelerations of x-axis, y-axis and z-axis from the 3-axis accelerometers.

In an embodiment of the third aspect, the set of motion sensors includes at least one gyroscope or 3-axis accelerometer.

In an embodiment of the third aspect, obtaining instantaneous physiological parameters according to physiological signal further comprises: obtaining accelerations of x-axis, y-axis and z-axis from the 3-axis accelerometer or gyroscope, the accelerations of x-axis, y-axis and z-axis are detected in a period time.

In an embodiment of the third aspect, transforming the movement data to physiological signal further comprises: transforming the accelerations of x-axis, y-axis and z-axis to inhale-to-exhale waveform by using digital signal processing techniques.

In an embodiment of the third aspect, obtaining instantaneous physiological parameters according to physiological signal further comprises: obtaining instantaneous breathing rate according to inhale-to-exhale waveform by using a preset algorithm, which includes a quality index that can be used to distinguish noisy waveform with good waveform.

In an embodiment of the third aspect, after obtaining instantaneous breathing rate according to inhale-to-exhale waveform by using a preset algorithm, the wristband further comprising instructions for: sending the instantaneous breathing rate to one or more health-related applications synchronously.

In accordance with a fourth aspect of the present invention, there is provided a smart terminal comprising a set of motion sensors for measuring movement of a user's body, at least one processor, at least one storage system in signal communication with the processor, one or more programs stored in the storage system and executable by the processor, the one or more programs comprising instructions for: obtaining movement data by using the set of motion sensors; transforming the movement data to physiological signal; obtaining instantaneous physiological parameters according to physiological signal.

In an embodiment of the fourth aspect, the set of motion sensors includes at least one gyroscope or 3-axis accelerometer, wherein obtaining movement data by using the set of motion sensors further comprises: obtaining accelerations of x-axis, y-axis and z-axis from the 3-axis accelerometer or gyroscope, the accelerations of x-axis, y-axis and z-axis are detected in a period time.

In an embodiment of the fourth aspect, transforming the movement data to physiological signal further comprises: transforming the accelerations of x-axis, y-axis and z-axis to inhale-to-exhale waveform by using digital signal processing techniques.

In an embodiment of the fourth aspect, obtaining instantaneous physiological parameters according to physiological signal further comprises: obtaining instantaneous breathing rate according to inhale-to-exhale waveform by using a preset algorithm, which includes a quality index that can be used to distinguish noisy waveform with good waveform.

In an embodiment of the fourth aspect, the set of motion sensors includes at least one 3-axis accelerometer, wherein obtaining movement data by using the set of motion sensors further comprises: obtaining accelerations of x-axis, y-axis and z-axis from the accelerometer, the accelerations of x-axis, y-axis and z-axis are detected in a period time.

In an embodiment of the fourth aspect, transforming the movement data to physiological signal further comprises: transforming the accelerations of x-axis, y-axis and z-axis to inhale-to-exhale waveform by using digital signal processing techniques.

In an embodiment of the fourth aspect, obtaining instantaneous physiological parameters according to physiological signal further comprises: obtaining instantaneous breathing rate according to inhale-to-exhale waveform by using a preset algorithm, which includes a quality index that can be used to distinguish noisy waveform with good waveform.

Other advantages and features will be apparent from the following detailed description when read in conjunction with the attached drawings.

### BRIEF DESCRIPTION OF THE DRAWINGS

Embodiments of the present invention will now be described, by way of example, with reference to the accompanying drawings in which:

FIG. 1 is a perspective view of a smart wristband, which may be used as a behaviour or an activity of an object, in accordance an embodiment of the present invention;

FIG. 2 is a block diagram of an embodiment of function module of the smart wristband of FIG. 1;

FIG. 3 is a flowchart of a method for analyzing a behavior or an activity of an object in accordance with an embodiment of the present invention;

FIG. 4 is plots showing a movement/motion data diagram obtained by the accelerometer in a smart wristband of FIG. 1;

FIG. 5 is an example inhales and exhales waveform obtained based on the movement data in FIG. 4;

FIG. 6 is a plot showing an instantaneous breathing rate obtained based on the waveform in FIG. 5; and

FIG. 7 is a block diagram of a system for analyzing a behavior or an activity of an object in accordance with an embodiment of the present invention.

It should be understand that the drawings are not necessarily to scale and that the disclosed embodiments may be sometimes illustrated diagrammatically and in partial views. In certain instances, details which are not necessary for an understanding of the invention, or which render other details difficult to perceive, may have been omitted. It should be understood, of course, that this disclosure is not limited to the particular embodiments illustrated herein.

### DETAILED DESCRIPTION OF THE PREFERRED EMBODIMENT

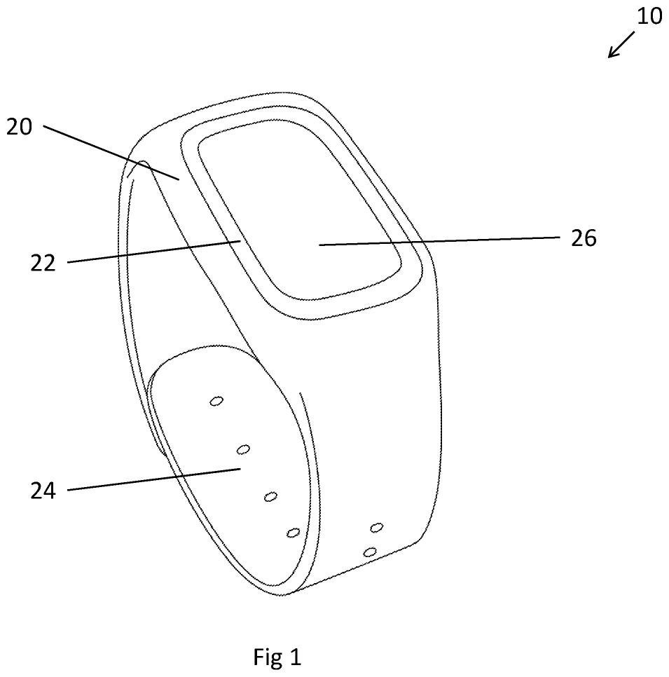

With reference to FIG. 1, there is shown an embodiment of a wearable device 20, such as a smart wristband or a smart watch. The wearable device 20 may be used as, wholly or partially, a system 10 for analyzing a behavior or an activity of an object (not shown). The system 10 comprises: a motion detection module arranged to obtain movement data associated with a motion of the object for a predetermined period of time; and a processing module arranged to process the movement data to obtain physiological parameters associated with the motion of the object, wherein the processing module is further arranged to determine a behavior or an activity of the object based on the obtained physiological parameters over the predetermined period of time.

In this embodiment, the system 10 is provided as a wearable device 20, which includes an engagement means, such as a device housing 22 and an adjustable wristband 24. The engagement means may be arranged to engage the motion detection module to an object, such that the motion of the object or a user may be sensed or detected by the motion detection module provided in the engagement means of the wearable device 20.

For example, the wearable device may be used as a smart wristband 20 which may be worn easily and conveniently, which means users are comfortable to wear wristbands continuously during daily activities. The smart wristband 20 may measure breathing rate based on the data obtained by the detection module in the wristband so that the data can be continuous measured conveniently and comfortably.

The material used for the wristband 24 itself can be any material that can be used to wrap around the wrist such as but not limited to plastic in this embodiment; it can also be metal pieces connecting each other with hinges or mechanisms alike. On either end of the wristband 24, there may be mechanism which can lock the other end of the wristband together tightly. In the present embodiment, there can be holes on one end of the wristband and mechanism like a pin that can penetrate through either one of the holes on the other end. With this mechanism, for example, users can wrap the wristband around one of the wrists and lock the band in place by piercing the pin through one of the desired holes.

In the course of use, the smart wristband 20 may also display different types of information to the user. For example, the aforesaid information may include but not limited to the instantaneous breathing rate, heart rate and so on and so forth. The wristband may also include a screen 26 for displaying the information such as but not limited to heart rate, breath rate and other details may be mounted or carried by the wristband in difference manners. Preferably, the screen 26 may be mounted atop the wristband by in the housing with an opening or transparent portion to visually exposing the screen to a user. The overall structure of the wristband 20 may be designed in a way that is suitable for the left-handed and the right-handed users.

Optionally or additionally, the wearable device may also comprise a communication module, such as a Bluetooth or a Wi-Fi communication module. The communication module may communicate data, such as the movement data collected by the detection module, to an external device such as a smartphone or a tablet computer or a remote computer server. The collected data may be further analyzed by a suitable processing module in an external device. Alternatively, the processing module may be locally included as a component in the wearable device.

At the same time with the presence of wireless connectivity (e.g. Wi-fi, Bluetooth etc.), the smart wristband 20 may send and exchange health data and other types data with other paired electronic devices.

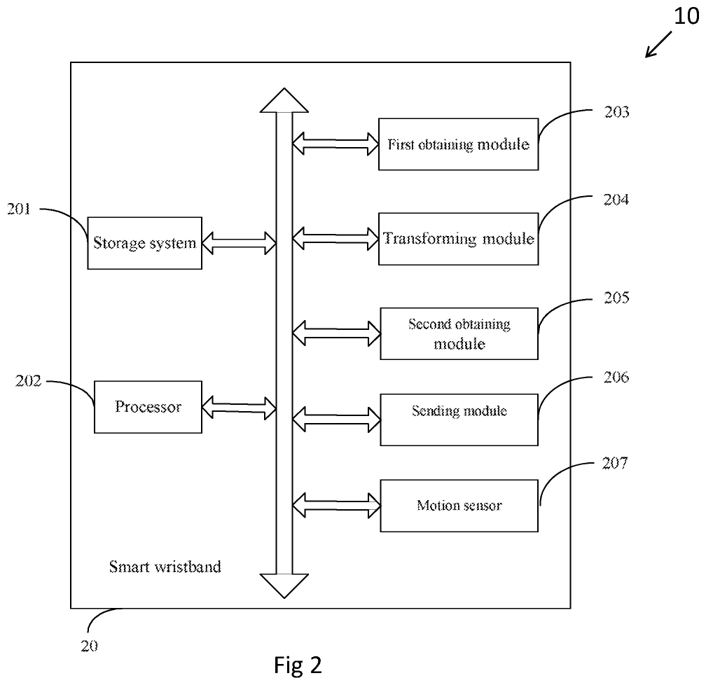

With reference to FIG. 2, the system 10 or the smart wristband 20 may also include a storage module or a storage system 201 arranged to at least temporally store the movement data obtained by the motion detection module, therefore the movement data may be transmitted externally for further processing later. The storage module may also computer instructions executable by a processor module 202, including a digital signal processor and/or a microprocessor such that the wristband perform all the necessary functions of the wristband 20.

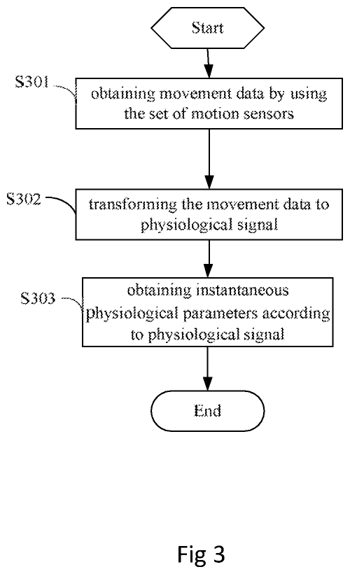

In this embodiment, the smart wristband 20 includes at least one storage system 201, at least one processor 202, a first obtaining module 203, a 15 transforming module 204, a second obtaining module 205, a sending module 206 and at least one motion sensor 207. The storage system 201 connects with the processor 202. The modules of the smart wristband 20 comprises one or more software programs in the form of computerized codes. The storage system 201 stores the computerized codes, and the processor 202 executes instructions of the computerized codes to provide functions for the 20 modules 203-206. The smart wristband 20 can execute the measuring physiological parameters method shown in FIG.3.

In addition, the smart wristband 20 further includes a motion detection module arranged to obtain movement data associated with a motion of the object for a predetermined period of time. The motion detection module may include one or more motion sensors 207 such as one or more single- or multi-axis accelerometers and/or gyroscopes. Preferably, the motion sensors may be high-precision multi-axis accelerometers which may sample the motion of the object with a sampling rate of at least 25 Hz and with a resolution of at least 12-bit. Alternatively, sensors or accelerometers with other specifications may be used based on different required precisions of the detection in other applications.

For example, a high-precision 3-axis accelerometer may in used to pick up 3-axis motion associated with the motion or the body movement of the user. The data may be transmitted either in real-time streamed wirelessly via Bluetooth protocol or recorded and stored on the wristband device and transmitted afterwards wirelessly via Bluetooth protocol.

Alternatively, the motion detection module may include a set of motion sensors, including at least one gyroscope or 3-axis accelerometer for detecting accelerations of x-axis, y-axis and z-axis from the 3-axis accelerometer or gyroscope during a period time. The processing module may then transform the accelerations of x-axis, y-axis and z-axis to inhale-to-exhale waveform by using digital signal processing techniques.

The processing module may include a first obtaining module 203 to obtain movement data by reading output data from the 3-axis accelerometer or gyroscope, the accelerations of x-axis, y-axis and z-axis are detected in a period time. The first obtaining module may also break and map out hand movement (e.g. swinging or other gestures) of the user wearing it, represented by 3-axises (X,Y,Z).

In addition, the processing module may include a transforming module 204 which may be used to transform the accelerations of x-axis, y-axis and z-axis to inhale-to-exhale waveform by using digital signal processing techniques. The physiological signal generated by the transforming module 204 may be but not limited to inhale and exhale rates.

For example, the transforming module 204 may transform the physiological signal to instantaneous physiological parameters by using digital signal processing techniques. This process may be carried out on the wristband device 20 or on other smart devices (e.g. personal computer, laptors, smart phones etc.). In the latter scenario, the wristband 20 may be communicated with the external processing unit through a radio frequency transmitter (e.g. Bluetooth, Wi-fi etc.).

Furthermore, the processing module may include a second obtaining module 205 obtains instantaneous breathing rate according to inhale-to-exhale waveform by using a preset algorithm. For example, second obtaining module may process the inhale-to-exhale waveform to identifies the number of inhalations and/or exhalations over a period of time during the sampling of movement data, thereby deriving a breathing rate of the user.

Optionally or additionally, the processing module is further arranged to filter a noise signal from the at least one source signal acquired by the motion sensor. The noise signal may be associated with a quality index of the at least one source signal acquired by the motion sensor. In one example, the quality index may be determined or assigned based on the intrinsic performance of the sensors 207 that may be used to distinguish noisy waveform with good waveform.

With reference also to FIG. 3, there is shown in example process steps of the method performed by the motion detection module and the processing module.

In S301, the first obtaining module 203 obtains movement data by using the set of motion sensors, such as accelerations of x-axis, y-axis and z-axis from the 3-axis accelerometers.

In S302, the transforming module 204 would then receive the movement data from the first obtaining module 203 and transform the aforesaid data to physiological signal. For example, the transforming module 204 may transforms the brief accelerations of x-axis, y-axis and z-axis from the 3-axis accelerometers to physiological signal. The second obtaining module 205 determines instantaneous inhale-to-exhale, heart rate, heart rate variability according to physiological signal.

In S303, the second obtaining module 205 obtaining instantaneous physiological parameters according to physiological signal. For example, the second obtaining module 205 may determine instantaneous inhale-to-exhale, heart rate, heart rate variability according to physiological signal.

Preferably, the physiological parameters may include an instantaneous breathing rate of the object. However, in other embodiments, the physiological parameters may be but be considered as a representation related to variation of electrical heart vector, heart work rate, respiratory rate, minutely respiratory volume gauge, temperature, systolic and diastolic blood pressure, and so on and so forth.

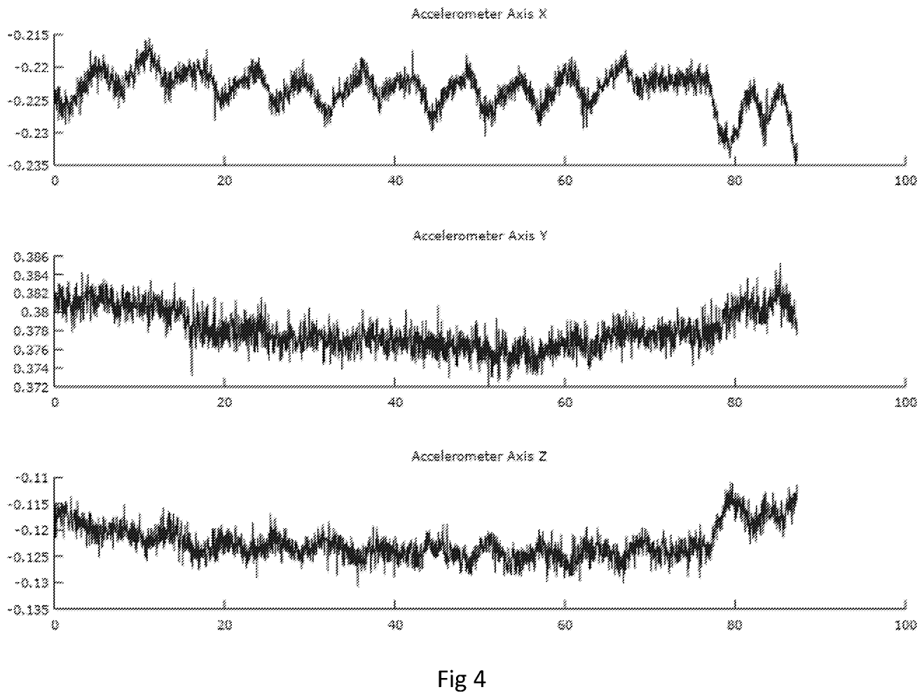

With reference to FIG. 4, there is shown movement data obtained in 3 independent axes including x-axis, y-axis and z-axis. It is also clearly shown that the fluctuation of signal, which may potentially representing hand movement, is relatively greater than that shown in y-axis and z-axis. The relatively larger fluctuation in the x-axis is generally caused by a body movement according to natural inhalations and exhalations of a user, in which the chest of the user may move up and down periodically and repeatedly. In this example, the x-axis may represent the vertical movement of the portion of body with the wristband 20 attached thereto.

Advantageously, the breakdown and graphical representation of movement into 3 independent axes may facilitate and simplify the analysis thereof for subsequent analysis.

Alternatively, the processing may further include a step of consolidating source signals associated with the detected motion obtained in a plurality of axes to obtain the movement data, as it is possible that the sensors may be oriented in a direction that the breathing movement is more obvious in a combined waveform based on two or more individual source signals obtained by the multi-axial motion sensors.

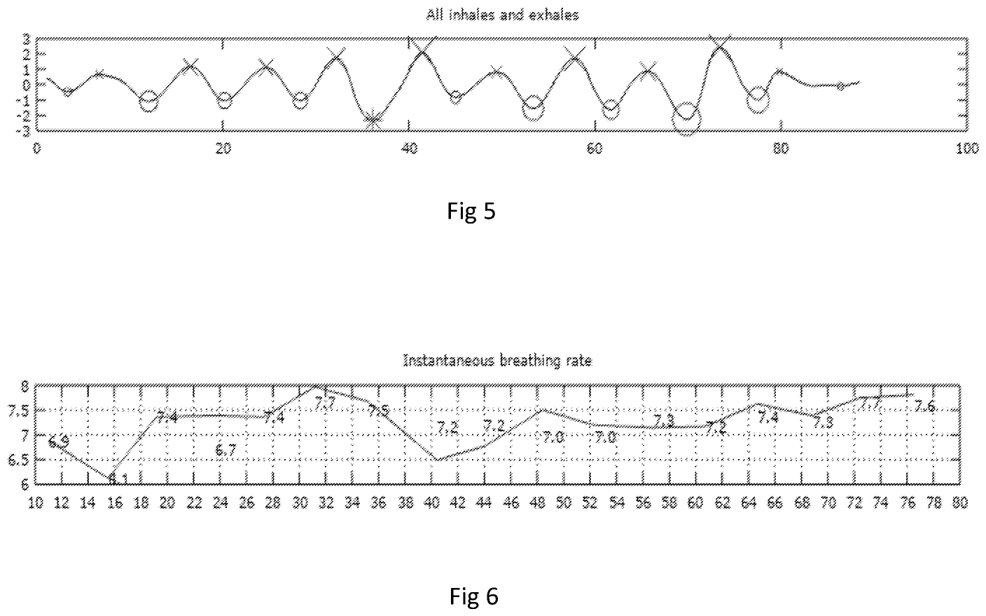

With reference to FIG. 5, there is shown a transformed inhales and exhales waveform based on the movement data detected, showing wave crest and wave trough. The peaks and valleys essentially refer to inhales and exhales respectively. For example, the crest which barely passes the “40-mark” mark implies the user at that point has a deep inhalation; whereas the trough locates at the “70-mark” implies the user at that point has a deep exhalation.

Referring to FIG. 6, there is shown a final result of the instantaneous breathing rate obtained based on the movement data obtained by the motion detection module. FIG. 6 shows instantaneous breathing rate curve, which shows instantaneous breathing rate in a period time, including a number of wave crest and wave trough across the time domain.

In a preferred example, the transforming module 204 may first remove the gravity factor from the 3-axis (X,Y,Z) data using either high-pass or detrend filter (X′,Y′,Z′). The processing unit may then extract the principle signal from the 3-axis data (X′,Y′,Z′) as shown in FIG. 4 and reduce the data to a single time-series (A) as shown in FIG. 5. The trend would then be removed from the time series (A) to a clean oscillating time series (A′). Then, a smoothing filter would be applied to the clean oscillating time series (A′) to the smoothed time series (B). The processor would then extract the peak and valley times (Pt, Vt) from the smoothed times series (B). At the end, the processing unit would then calculate a series of breathing rates.

After obtaining instantaneous breathing rate according to inhale-to-exhale waveform by using a preset algorithm, the sending module 206 may send the instantaneous breathing rate, heart rate, heart rate variability, to one or more health-related applications synchronously, so that many more health-related applications can analyze the instantaneous breathing rate, heart rate, heart rate variability clearly.

Optionally, the breathing rate and/or other instantaneous physiological parameters according to physiological signal, may be further processed or transmitted using the sending module 206, e.g. to the health-related applications.

The sending could be done in the presence of wireless connectivity. The wireless connectivity may include but not limited to Wi-fi, Bluetooth and so on and so forth. Optionally, upon detecting any irregular health condition, the wristband may immediately alert relevant authorities.

With reference to FIG. 6, the crest located at the “32 mark” in the curve showing the instantaneous breathing rate may imply the user has a relatively high breathing rate as compared to the rest of the time. In turn, this could imply the user could be mentally and/or physiologically active. By a similar token, the trough located at the “16 mark” may imply that the user has a relatively low breathing rate as compared to the rest of the time. In other words, this could mean that the user is relaxing.

Advantageously, such ability to send the health information and alert to health-related applications and relevant authorities instantaneously could help authorities to better monitor the health condition of the user. In addition, in case of emergencies, health-related applications or devices and authorities could take prompt reaction in response to the alert sent by the device.

To initialize the wristband for operating, the user may switch on the device through some switching mechanism (e.g. electrical or mechanical). Such mechanism may be located on either one of the four side of the panel. For the first time of use, the device may prompt the user to register an account either directly on the wristband or on other electronic device (e.g. laptop, tablets etc.). In the latter case, the wristband itself may communicate with the internet by some radio frequency identification method such as Wi-fi.

Optionally, the user may hook up the wristband with other electronic devices (e.g. smart phones, electronic tablets, personal computers). The wristband may be communicable with other devices through some radio frequency identification technology (e.g. Wi-fi, Bluetooth etc.). Advantageously, the ability to communicate with other electronic devices may allow the device to wirelessly transmit data (e.g. health record, location etc.) to the paired electronic device, thus it promotes convenient data exchange and storage.

To begin the health monitoring function, the user is required to firstly wear the wristband on either left or right wrist and secure the band with its locking mechanism (e.g. buckle, valcro strap, strap buckle and so on and so forth). The user may then have to turn on the device and select the health monitoring function in the device. Upon successful initiation of the function, the device may send a notification regarding so to the user on the screen of the wristband or other electronic tied with it.

For example, if the user is wearing it to monitor the health condition, the wristband may instantaneously generate an instantaneous breathing rate chart when the processor observes a predetermined pattern from the movement data obtained by the accelerometers. For example, the breathing rate of the user may be measured during static activity like sitting, sleeping, meditation, etc. The wearable device may use this data to evaluate the mental state of a user, e.g. a high breathing rate may imply a busy mind, mind wandering, mentally exhausted, etc.

With reference to FIG. 6, the x-axis of this chart represents time in second; whereas the y-axis represents the breathing rate. At “16 second”, the user might be mentally relaxed and therefore the breathing rate is relatively lower. In contrast, at “32 second”, the user might be nervous or mentally busy and therefore the breathing rate is relatively higher than the rest of the time.

The device may be put on sleep mode manually or automatically after a certain period of idling. If the user wishes to terminate the function, the user may turn on the device and click on a function key to kill the health monitoring function.

The power for the electronic portion of the wristband may be supplied by battery. The power may be rechargeable by various means including but not exhaustive by wireless charging and direct charging by cable.

These embodiments may be advantageous in that physiological parameters such as breathing rate of a user may be obtained or estimated based on the body movement of the user, in which the body movement may be readily detected using high-precision accelerometer and the discussed methods to process the raw movement data obtained from the accelerometer.

Advantageously, by instantaneously estimating the breathing rate of a user, the behavior or the activity of the user may be easily monitored

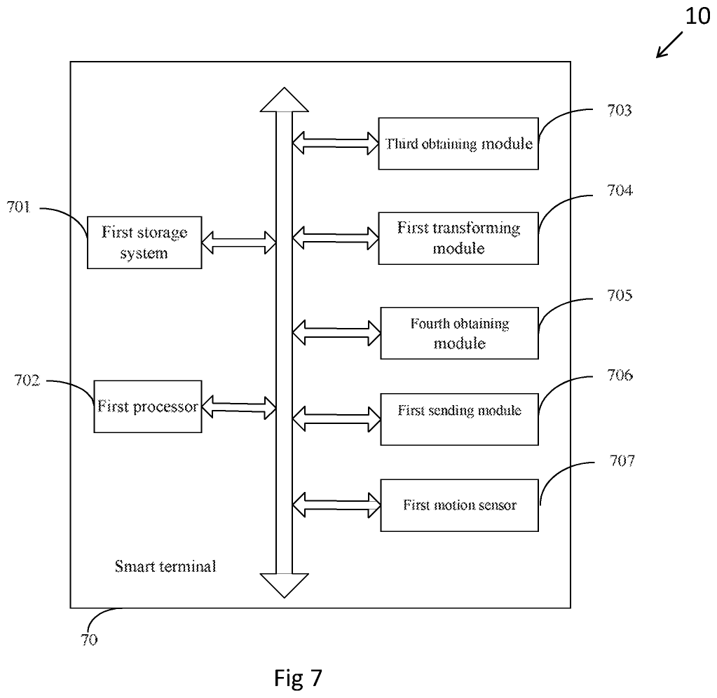

Referring to FIG. 7 there is shown an alternative embodiment of the present invention, which may be implemented as smart terminal 70 including at least one first storage system 701, at least one first processor 702, a third obtaining module 703, a first transforming module 704, a fourth obtaining module 705, a first sending module 706 and at least one first motion sensor 707. The first storage system 701 connects with the first processor 702. The modules of the smart terminal 70 comprises one or more software programs in the form of computerized codes. The first storage system 701 stores the computerized codes, and the first processor 702 executes instructions of the computerized codes to provide functions for the modules 703-706. In this embodiment, the third obtaining module 703 obtains movement data by using the set of motion sensors. The first transforming module 704 transforms the movement data to physiological signal. The fourth obtaining module 705 obtaining instantaneous physiological parameters according to physiological signal.

In this embodiment, the set of motion sensors includes at least one gyroscope or 3-axis accelerometer, the third obtaining module 703 obtains accelerations of x-axis, y-axis and z-axis from the 3-axis accelerometer or gyroscope, the accelerations of x-axis, y-axis and z-axis are detected in a period time. The first transforming module 704 transforms the accelerations of x-axis, y-axis and z-axis to inhale-to-exhale waveform by using digital signal processing techniques. The fourth obtaining module 705 obtains instantaneous breathing rate according to inhale-to-exhale waveform by using a preset algorithm, which includes a quality index that can be used to distinguish noisy waveform with good waveform.

In this embodiment, the set of motion sensors includes at least one 3-axis accelerometer, the third obtaining module 703 obtains accelerations of x-axis, y-axis and z-axis from the accelerometer, the accelerations of x-axis, y-axis and z-axis are detected in a period time; the first transforming module 704 transforms the accelerations of x-axis, y-axis and z-axis to inhale-to-exhale waveform by using digital signal processing techniques; the fourth obtaining module 705 obtains instantaneous breathing rate according to inhale-to-exhale waveform by using a preset algorithm, which includes a quality index that can be used to distinguish noisy waveform with good waveform.

Preferably, the smart terminal 70 can execute the disclosed functions of the smart wristband 20. The smart terminal 70 can be a smart phone. For example, the smart phone may process movement data obtained by the first motion sensor 707, with or without additional movement data from the smart wristband 20, so as to determine one or more physiological parameters of the user.

It will also be appreciated that where the methods and systems of the present invention are either wholly implemented by computing system or partly implemented by computing systems then any appropriate computing system architecture may be utilised. This will include stand alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A system for analyzing a behavior or an activity of an object, comprising:
a motion detection module arranged to obtain movement data associated with a motion of the object for a predetermined period of time; and
a processing module arranged to process the movement data to obtain physiological parameters associated with the motion of the object,
wherein the processing module is further arranged to determine a behavior or an activity of the object based on the obtained physiological parameters over the predetermined period of time.

15. The system for analyzing a behavior or an activity of an object in accordance with claim 14, wherein the physiological parameters includes an instantaneous breathing rate of the object.

16. The system for analyzing a behavior or an activity of an object in accordance with claim 15, wherein the motion of the object includes a body movement as a result of breathing of the object.

17. The system for analyzing a behavior or an activity of an object in accordance with claim 16, wherein the motion of the object is represented as an inhale-to-exhale waveform associated with the movement data.

18. The system for analyzing a behavior or an activity of an object in accordance with claim 17, wherein the inhale-to-exhale waveform includes peaks and valleys each representing an inhalation and an exhalation respectively.

19. The system for analyzing a behavior or an activity of an object in accordance with claim 14, wherein the motion detection module comprises at least one motion sensor including at least one of an accelerometer, a multi-axis accelerometer, a gyroscope, and a multi-axis gyroscope.

20. (canceled)

21. (canceled)

22. (canceled)

23. The system for analyzing a behavior or an activity of an object in accordance with claim 19, wherein the accelerometer or the multi-axis accelerometer is arranged to sample the motion of the object with a sampling rate of at least 25 Hz.

24. The system for analyzing a behavior or an activity of an object in accordance with claim 19, wherein the accelerometer or the multi-axis accelerometer is arranged to sample the motion of the object with a resolution of at least 12-bit.

25. The system for analyzing a behavior or an activity of an object in accordance with claim 19, wherein the processing module is further arranged to process at least one source signal obtained by the at least one motion sensor associated with the detected motion to obtain the physiological parameters.

26. The system for analyzing a behavior or an activity of an object in accordance with claim 25, wherein the processing module is further arranged to filter a noise signal from the at least one source signal acquired by the motion sensor.

27. The system for analyzing a behavior or an activity of an object in accordance with claim 26, wherein the noise signal is associated with a quality index of the at least one source signal acquired by the motion sensor.

28. The system for analyzing a behavior or an activity of an object in accordance with claim 26, wherein the processing module is further arranged to consolidating source signals associated with the detected motion obtained in a plurality of axes to obtain the movement data.

29. The system for analyzing a behavior or an activity of an object in accordance with claim 14, wherein the physiological parameters further include a heart rate and/or a heart rate variability of the object.

30. The system for analyzing a behavior or an activity of an object in accordance with claim 14, further comprising an engagement means arranged to engage the motion detection module to an object or a user.

31. (canceled)

32. The system for analyzing a behavior or an activity of an object in accordance with claim 30, further comprising a communication module arranged to communicate the movement data to an external device including the processing module.

33. The system for analyzing a behavior or an activity of an object in accordance with claim 14, further comprising a storage module arranged to at least temporally store the movement data obtained by the motion detection module.

34. (canceled)

35. (canceled)

36. A method for analyzing a behavior or an activity of an object, comprising the steps of:
obtaining movement data associated with a motion of the object for a predetermined period of time;
processing the movement data to obtain physiological parameters associated with the motion of the object; and
determining a behavior or an activity of the object based on the obtained physiological parameters over the predetermined period of time.

37. The method for analyzing a behavior or an activity of an object in accordance with claim 36, wherein the physiological parameters includes an instantaneous breathing rate of the object.

38. The method for analyzing a behavior or an activity of an object in accordance with claim 37, wherein the motion of the object includes a body movement as a result of breathing of the object.

39. The method for analyzing a behavior or an activity of an object in accordance with claim 38, wherein the motion of the object is represented as an inhale-to-exhale waveform associated with the movement data.

40. The method for analyzing a behavior or an activity of an object in accordance with claim 39, wherein the inhale-to-exhale waveform includes peaks and valleys each representing an inhalation and an exhalation respectively.

41. The method for analyzing a behavior or an activity of an object in accordance with claim 36, further comprising the step of detecting the motion of the object using at least one motion sensor, including at least one of an accelerometer, a 3-axis accelerometer, a gyroscope and a 3-axis gyroscope.

42. The method for analyzing a behavior or an activity of an object in accordance with claim 41, further comprising the step of processing at least one source signal obtained by the at least one motion sensor associated with the detected motion to obtain the physiological parameters.

43. The method for analyzing a behavior or an activity of an object in accordance with claim 42, wherein the step of processing the at least one source signal includes filtering a noise signal from the at least one source signal acquired by the motion sensor.

44. The method for analyzing a behavior or an activity of an object in accordance with claim 43, wherein the noise signal is associated with a quality index of the at least one source signal acquired by the motion sensor.

45. The method for analyzing a behavior or an activity of an object in accordance with claim 43, further comprising the step of consolidating source signals associated with the detected motion obtained in a plurality of axes to obtain the movement data.

46. The method for analyzing a behavior or an activity of an object in accordance with claim 36, wherein the physiological parameters further include a heart rate and/or a heart rate variability of the object
